(19) United States
(12) Patent Application Publication
(10) Pub. No.: US 2017/0042809 A1
(43) Pub. Date: Feb. 16, 2017

(54) STABLE LIPOSOMAL FORMULATIONS OF CARBONIC ANHYDRASE INHIBITORS FOR OCULAR DRUG DELIVERY

(71) Applicant: Peregrine Ophthalmic PTE LTD., Singapore (SG)

(72) Inventors: Ting-Bin Yu, Irvine, CA (US); Zhiwei Shi, Irvine, CA (US); Shih-Horng Su, Irvine, CA (US); Tina Howden, Singapore (SG)

(21) Appl. No.: 14/823,692

(22) Filed: Aug. 11, 2015

(51) Int. Cl. A61K 9/127; A61K 47/24; A61K 31/542; A61K 31/382; A61K 31/433

(52) U.S. Cl. CPC A61K 9/127; A61K 31/382; A61K 31/433; A61K 31/542; A61K 47/24

(57) ABSTRACT

A stable liposomal formulation for ocular delivery of a carbonic anhydrase inhibitor. The formulation contains (i) a liposome that includes a lipid bilayer formed of a phosphatidylcholine, and (ii) a carbonic anhydrase inhibitor encapsulated in the liposome. Also provided is a method for treating an ocular disorder with the formulation.

### BACKGROUND OF THE INVENTION

Field

This application relates to stable formulations for ocular delivery of carbonic anhydrase inhibitors.

Background Information

Elevated intraocular pressure (IOP) is one of the major risk factors in the pathogenesis of optic nerve damage and glaucomatous visual field loss. IOP is influenced by the balance between inflow and outflow of aqueous humour from the eye. As such, inhibition of aqueous humour secretion is one common strategy to reduce IOP.

Carbonic anhydrase II (CA-II) is the main carbonic anhydrase isoenzyme involved in aqueous humor secretion. Inhibiting human CA-II in the ciliary processes of the eye decreases aqueous humor secretion. Dorzolamide, an inhibitor of human CA II, has been used extensively to reduce elevated IOP by topical ocular administration. Typically, dorzolamide is administered three times daily. This regimen has many drawbacks, including ocular allergy from repeated drug administration and poor patient compliance. Poor compliance leads to suboptimal control of IOP and disease progression eventually leading to blindness.

Delivery of drugs in a sustained manner for treating IOP would give patients a better alternative by providing an optimal therapeutic index with minimal toxicity and inconvenience. In this regard, vesicles containing synthetic non-ionic surfactants, e.g., sorbitan monostearates and sorbitan trioleate, have been used to improve ocular delivery of dorzolamide. See Hasan et al., Pharm. Dev. Technol. 19(6):748-754. However, the synthetic ingredients pose a potential toxicity risk through repeated applications over a long course of treatment.

The need exists to develop new safer biocompatible formulations of CAII inhibitors to prolong drug delivery and improve drug stability, leading to a higher therapeutic index for these inhibitors.

### SUMMARY

To address the need for improved delivery of carbonic anhydrase inhibitors, a stable liposomal formulation for ocular delivery is provided.

The formulation includes a liposome that contains at least one lipid bilayer. The lipid bilayer contains a phosphatidylcholine. The liposome has a diameter of less than 2 μm and is free of cholesterol and non-ionic detergents. A carbonic anhydrase inhibitor is encapsulated in the liposome. The weight ratio between the carbonic anhydrase inhibitor and the phosphatidylcholine is 1:10 to 1:100.

Also provided is a method for treating an ocular disorder by administering to an eye of a subject in need of the treatment the stable liposomal formulation described above.

The details of one or more embodiments of the invention are set forth in the drawing and description below. Other features, objects, and advantages of the invention will be apparent from the description and from the claims.

All references cited herein are hereby incorporated by reference in their entirety.

### BRIEF DESCRIPTION OF THE DRAWINGS

The invention description below refers to the accompanying drawing, of which:

FIG. 1 is a plot of the in vitro release profiles of dorzolamide HCl (DH) and liposomal dorzolamide (LipoDH).

### DETAILED DESCRIPTION

As mentioned above, a stable liposomal formulation for ocular delivery is disclosed. The formulation includes a carbonic anhydrase inhibitor encapsulated in the liposome. The carbonic anhydrase inhibitor can be dorzolamide, acetazolamide, brinzolamide, and methazolamide or a combination of these inhibitors. In a particular aspect, the carbonic anhydrase inhibitor is dorzolamide.

The liposome has a diameter of less than 2 μm. In an embodiment, the liposome is at least 50 nm in diameter and less than 2 μm in diameter. For example, the diameter can be 50 nm, 100 nm, 150 nm, 200 nm, 250 nm, 300 nm, 350 nm, 400 nm, 500 nm, 1 μm, or 1.5 μm. Preferably, the diameter of the liposome is between 100 nm and 400 nm. In a particular embodiment, the diameter of the liposome is 150 nm. In a preferred embodiment, the liposome is a large unilamellar vesicle (LUV) having the just-mentioned diameters.

The liposome, as mentioned above, contains at least one lipid bilayer including a phosphatidylcholine and is free of cholesterol and non-ionic detergents. The phosphatidylcholine can be one or more of egg phosphatidyleholine (EggPC); palmitoyl oleoyl phosphatidylcholine (POPC); 1,2-dimyristoyl-sn-glycero-3-phosphocholine (DMPC); and 1,2-dioleoyl-sn-glycero-3-phosphocholine (DOPC).

To reiterate, a carbonic anhydrase inhibitor is encapsulated in the liposome. The weight ratio between the carbonic anhydrase inhibitor and the phosphatidylcholine can be from 1:10 to 1:100.

In addition to the phosphatidylcholine, the liposome can further include a phospholipid conjugated to a polyethylene glycol (PEG) moiety. The PEG-conjugated phospholipid can be 1,2-distearoyl-sn-glycero-3-phosphoethanolamine-N-[amino(PEG)]; 1,2-dioleoyl-sn-glycero-3-phosphoethanolamine-N-[methoxy(PEG)]; 1,2-dipalmitoyl-sn-glycero-3-phosphoethanolamine-N-[methoxy(PEG)]; 1,2-dimyristoyl-sn-glycero-3-phosphoethanolamine-N-[methoxy(PEG)]; or mixtures thereof.

The molecular weight of the PEG moiety can be 350 to 3000 g/mol, e.g., 350, 500, 750, 1000, 1250 1500, 1750, 2000, 2250, 2500, 2750, 3000 g/mol. In a preferred embodiment, the molecular weight of the PEG moiety is 2000 g/mol.

In a particular aspect, the stable liposomal formulation includes dorzolamide encapsulated in a liposome that contains POPC and 1,2-distearoyl-sn-glycero-3-phosphoethanolamine-N-[amino(PEG)], where the PEG moiety has a molecular weight of 2000 g/mol, the weight ratio between the dorzolamide and the POPC is 1:10 to 1:30, the liposome has a diameter of 150 nm and is free of cholesterol and non-ionic detergents.

The stable liposomal formulations can be an aqueous formulation, e.g., a suspension. In a particular embodiment, the stable liposomal formulations are lyophilized formulations.

Liposomes loaded with the carbonic anhydrase inhibitors, e.g., dorzolamide, acetazolamide, brinzolamide, and methazolamide, can be produced via a thin-film hydration method. In brief, a thin film containing both the carbonic anhydrase inhibitor and the phosphatidylcholine and, optionally, the PEG-conjugated phospholipid, is formed through solvent evaporation. The solvent can be methanol, chloroform, ethanol, or mixtures thereof. In a particular aspect, the solvent is methanol. The thin film, having a thickness of 0.1 to 1500 μm, is hydrated in phosphate-buffered saline (PBS) to form multi-lamellar vesicles (MLVs). Subsequently, the MLVs can be converted into LUVs by an extrusion process. For example, the MLVs can be extruded through a polycarbonate filter from 3 to 20 times. In a preferred extrusion process, the MLVs are extruded 10 times.

The filter can have a pore size ranging from 50 nm to 200 nm. In a particular embodiment, the pore size is 100 nm. Again, the resulting LUV, i.e., liposome, can have a diameter of less than 2 μm (e.g., 50 nm, 100 nm, 150 nm, 200 nm, 250 nm, 300 nm, 350 nm, 400 nm, 500 nm, 1 μm, and 1.5 μm).

The weight ratio of carbonic anhydrase to phosphatidylcholine in the liposome can range from 1:10 to 1:100. In an embodiment, the weight ratio is 1:10 to 1:50. In a specific embodiment, the ratio is 1:10.

The stable liposomal formulations described above can have a carbonic anhydrase content of 1 mg/mL to 50 mg/mL. In an embodiment, the stable liposomal formulation has a carbonic anhydrase content of 10 mg/mL to 50 mg/mL. In a particular aspect, the carbonic anhydrase content is 10 mg/mL, a concentration comparable to that of commercially available eyedrops containing carbonic anhydrase inhibitors.

The stable liposomal formulations also improve the stability of the carbonic anhydrase inhibitors in solution. For example, the carbonic anhydrase inhibitors in the formulation can be stable for 7-14 days when stored at 5° C. as compared to the inhibitor in an aqueous solution. In this context, stability is defined as a loss of no more than 20% of the starting amount of drug in the formulation.

Advantageously, the carbonic anhydrase inhibitors can be released from the liposomes in the formulation over an extended period of time after administration. That is to say, the formulation is a sustained release formulation. For example, the carbonic anhydrase inhibitors can be released from the liposomes after administration of the formulation continuously over a period of up to 3 months, e.g., 7, 14, 21 days and 1, 2, and 3 months.

The stable liposomal formulations are preferably prepared for ocular delivery. For example, the stable liposomal formulations can be in the form of eye drops. In a preferred embodiment, the stable liposomal formulations are in the form of an injectable solution.

In an embodiment, the stable liposomal formulations are lyophilized formulations. The lyophilized formulations can be stored for extended periods in the dry state and is hydrated as needed to form, e.g., eye drops and injectable solutions.

As mentioned above, a method for treating an ocular disorder by administering the stable liposomal formulations is also disclosed. Any of the above-described stable liposomal formulations can be administered to a subject suffering from an ocular disorder. The subject is identified by techniques known in the art. The ocular disorder can be, e.g., ocular hypertension or glaucoma. In a particular aspect, the glaucoma is open-angle glaucoma.

Treatment of an ocular disorder can be accomplished by administering eye drops containing the disclosed stable liposomal formulations. In a preferred route, the stable liposomal formulations are administered via subconjunctival injection.

The stable liposomal formulations that can be used in the method for treating an ocular disorder include a carbonic anhydrase inhibitor encapsulated in a liposome. The carbonic anhydrase inhibitor can be dorzolamide, acetazolamide, brinzolamide, and methazolamide or a combination of these inhibitors.

The liposome in the stable liposomal formulation that can be used in the method for treating an ocular disorder has a diameter of less than 2 μm. In an embodiment, the liposome is at least 50 nm in diameter and less than 2 μm in diameter. For example, the diameter can be 50 nm, 100 nm, 150 nm, 200 nm, 250 nm, 300 nm, 350 nm, 400 nm, 500 nm, 1 μm, or 1.5 μm. Preferably, the diameter of the liposome is between 100 nm and 400 nm. In a particular embodiment, the diameter of the liposome is 150 nm. In a preferred embodiment, the liposome is an LUV having the just-mentioned diameters.

The liposome in the stable liposomal formulation that can be used in the method for treating an ocular disorder, as mentioned above, contains at least one lipid bilayer including a phosphatidylcholine and is free of cholesterol and non-ionic detergents. The phosphatidylcholine can be one or more of egg phosphatidylcholine (EggPC); palmitoyl oleoyl phosphatidylcholine (POPC); 1,2-dimyristoyl-sn-glycero-3-phosphocholine (DMPC); and 1,2-dioleoyl-sn-glycero-3-phosphocholine (DOPC).

To reiterate, a carbonic anhydrase inhibitor is encapsulated in the liposome in the stable liposomal formulation that can be used in the method for treating an ocular disorder. The weight ratio between the carbonic anhydrase inhibitor and the phosphatidylcholine can be from 1:10 to 1:100.

In addition to the phosphatidylcholine, the liposome in the stable liposomal formulation that can be used in the method for treating an ocular disorder can further include a phospholipid conjugated to a polyethylene glycol (PEG) moiety. The PEG-conjugated phospholipid can be 1,2-distearoyl-sn-glycero-3-phosphoethanolamine-N-[amino(PEG)]; 1,2-dioleoyl-sn-glycero-3-phosphoethanolamine-N-[methoxy(PEG)]; 1,2-dipalmitoyl-sn-glycero-3-phosphoethanolamine-N-[methoxy(PEG)]; 1,2-dimyristoyl-sn-glycero-3-phosphoethanolamine-N-[methoxy(PEG)]; or mixtures thereof.

The molecular weight of the PEG moiety can be 350 to 3000 g/mol, e.g., 350, 500, 750, 1000, 1250 1500, 1750, 2000, 2250, 2500, 2750, 3000 g/mol. In a preferred embodiment, the molecular weight of the PEG moiety is 2000 g/mol.

In a particularly preferred embodiment, the method for treating an ocular disorder entails treating an open-angle glaucoma patient by injecting subconjunctivally a stable liposomal formulation of dorzolamide, where the liposome includes POPC and 1,2-distearoyl-sn-glycero-3-phosphoethanolamine-N-[amino(PEG-2000)], the weight ratio between the dorzolamide and the POPC is 1:10 to 1:30, and the liposome has a diameter of 150 nm.

Without further elaboration, it is believed that one skilled in the art can, based on the description above, utilize the present invention to its fullest extent. The specific examples below are to be construed as merely illustrative, and not limitative of the remainder of the disclosure in any way whatsoever.

### EXAMPLES

### Example 1

### Preparation of Dorzolamide-Loaded Liposomes Containing EggPC, DMPC, or DOPC

Three 100 mg samples of dorzolamide HCl were each mixed separately with 2000 mg of EggPC, DMPC, or DOPC in methanol in a 20 mL depyrogenated glass bottle to form three 10 ml solutions. For each solution, 1 mL of ethanol was added and the solution was stirred until it became clear. A nitrogen gas stream was used to evaporate the alcohols at room temperature for at least 90 min. to form a transparent thin film. 1 mL of PBS at pH 7.4 was added to the film and stirred for at least 30 min., thereby forming multilamellar vesicles (MLVs). The sizes of the MLVs were reduced by extrusion through a 3-stack of polycarbonate filter membranes (pore size 100 nm) using a bench top extruder (Northern Lipids Inc., Canada). After 10 extrusion passes, large unilamellar vesicles (LUVs) with an average size of ˜160 nm were obtained. The dorzolamide-loaded liposomes were analyzed for drug content, mean vesicle size, and polydispersity. The physical characteristics of the dorzolamide-loaded liposomes were determined essentially as described in Venkatraman et al., International Application Publication No. 2012/021107, the content of which is incorporated herein by reference in its entirety. The results are shown in Table 1 below.

TABLE 1
Physical properties of dorzolamide-loaded liposomes formed of EggPC,
DMPC, and DOPC

| | Liposome | | |
|---|---|---|---|
| Properties | EggPC | DMPC | DOPC |
| drug content (mg/mL) | 6.76 | 8.50 | 8.25 |
| PC content (mg/mL) | 180 | 36 | 82.6 |
| drug-to-lipid ratio | 1:26.6 | 1:4.24 | 1:10.0 |
| mean vesicle size (nm) | 180.0 | 133.7 | 123.6 |
| polydispersity index (PDI) | 0.545 | 0.241 | 0.113 |

### Example 2

### Preparation of Dorzolamide-Loaded Liposomes Containing PEG-Ylated Phospholipid LUVs

600 mg of dorzolamide HCl, 11.4 g of POPC, and 600 mg 1,2-distearoyl-sn-glycero-3-phosphoethanolamine-N-[amino(polyethylene glycol)-2000] (ammonium salt) in 50 mL of methanol was added to a 250 mL depyrogenated glass bottle. After adding 25 mL ethanol, the solution was stirred until clear. A nitrogen gas stream was used to evaporate the alcohols at room temperature for at least 90 min. to form a transparent thin film. 50 mL PBS at pH 7.4 was added to the film and stirred for at least 30 min., thereby forming multilamellar vesicles (MLVs). The sizes of the MLVs were reduced by extrusion through a 3-stack of polycarbonate filter membranes (pore size 100 nm) using a bench top extruder (Northern Lipids Inc., Canada). After 10 extrusion passes, large unilamellar vesicles (LUVs) with an average size of ˜150 nm were obtained. The dorzolamide-loaded PEGylated liposomal formulation (“LipoDH”) was then analyzed for the drug content, mean vesicle size, and polydispersity as described above in EXAMPLE 1. The results are shown in Table 2 below.

TABLE 2
Physical properties of dorzolamide-loaded PEGylated Liposomes

| Properties | Measured Values |
|---|---|
| dorzolamide content | 10.7 mg/mL |
| POPC content | 188.0 mg/mL |
| dorzolamide to POPC ratio | 1:17.6 |
| mean vesicle size | 146 nm |
| PDI | 0.175 |

### Example 3

### Stability of Dorzolamide-Loaded Liposomes Containing PEGylated Phospholipid LUVs (LipoDH)

The stability of dorzolamide loaded liposomes was compared to dorzolamide HCl formulated in an aqueous solution. The LipoDH formulation was described above in EXAMPLE 2. A dorzolamide HCl aqueous solution was prepared by dissolving 200 mg dorzolamide HCl in 20 mL of PBS. The dorzolamide content of both formulations was analyzed after storing them at 5° C. for 7 and 14 days. The temperature was monitored and recorded continuously to ensure stable temperature conditions. The results are shown in TABLE 3 below.

TABLE 3
Stability of LipoDH and dorzolamide HCl in an aqueous solution.

| | | Start of Stability | 7 Days | 14 Days |
|---|---|---|---|---|
| Group | Properties | Test | Incubation | Incubation |
| LipoDH | drug content | 10.7 | 11.2 | 8.52 |
| | (mg/mL) | | | |
| | degradation | 0.0% | 0.0% | 20.3% |
| | percentage | | | |
| dorzolamide | drug content | 9.5 | 4.0 | 3.9 |
| HCl | (mg/mL) | | | |
| | degradation | 0.0% | 57.9% | 58.9% |
| | percentage | | | |

The stability results indicated that the liposomal dorzolamide solution is stable for at least 7 days upon storage at 5° C. while at the same time the drug content of a dorzolamide HCl aqueous solution decreases to 57.9% at 7 days. Clearly, the liposomal formulation significantly improves the stability of dorzolamide.

### Example 4

### Measurement of Dorzolamide-HCl Encapsulation Efficiency

Dorzolamide-loaded liposomes were prepared as described above in EXAMPLE 1 and EXAMPLE 2. The encapsulation efficiency of dorzolamide-HCl in the different liposomes was evaluated by employing gel-filtration to remove free dorzolamide-HCl from the liposomal preparations. The drug to lipid ratio of liposomal formulation samples were determined before and after running them on a PD-10 cross-linked dextran gel (SEPHADEX® G-25) desalting column using the following equation:

$$Drug Encapsulation \% = \frac{\begin{matrix}Final Drug \text{:} Lipid Ratio \\ ( sample passed through PD \text{-} 10 column )\end{matrix}}{\begin{matrix}Initial Drug \text{:} Lipid Ratio \\ ( samples prior to PD \text{-} 10 separation )\end{matrix}} \times 100 \%$$

The drug encapsulation efficiency results are summarized in TABLE 4 below.

TABLE 4
Drug encapsulation efficiency of dorzolamide-loaded liposomes

| | Drug:Lipid Ratio | Drug:Lipid Ratio | Drug |
|---|---|---|---|
| Liposome | prior to column | after column | Encapsulation |
| Formulation | separation | separation | Efficiency |
| EggPC | 1:26.6 | 1:41.7 | 64% |
| DMPC | 1:4.23 | 1:50.0 | 8.4% |
| DOPC | 1:10.1 | 1:47.6 | 21% |
| LipoDH | 1:17.6 | 1:28.4 | 62.0% |

### Example 5

### In-Vitro Drug Release Study

Drug release studies were performed by dialysing a dorzolamide HCl solution and LipoDH prepared as described above in EXAMPLE 2 and EXAMPLE 3, respectively, against PBS at a pH of 7.4 and measuring the amount of dorzolamide released by HPLC.

Briefly, 1 mL of dorzolamide HCl solution and 1 ml LipoDH were each loaded into a dialysis tube (molecular weight cut-off of 8-10 kDal) before placing the tube into 40 mL of PBS in a 50 mL tube. The PBS in the tubes was sampled at 1, 4, 7, 24, 48, 72 and 96 hours at which time the dorzolamide concentration was measured by HPLC.

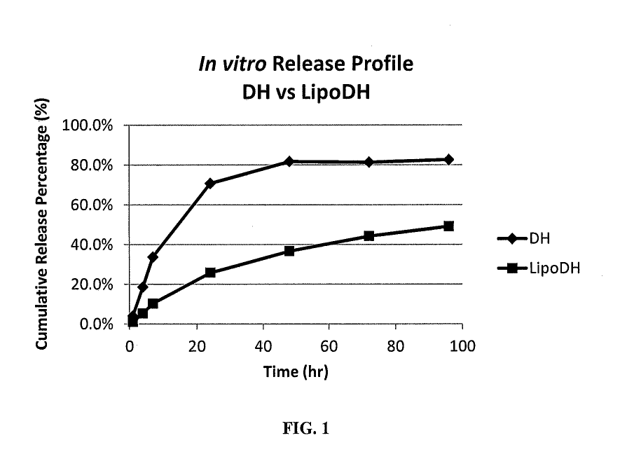

The cumulative drug release percentage vs release time for both dorzolamide HCl solution (unencapsulated) and LipoDH (encapsulated) was calculated and plotted. The results, shown in FIG. 1, indicated that 80% of the unencapsulated dorzolamide diffused through the dialysis tubing within 48 hours while, at the same time, only ˜37% of the encapsulated dorzolamide was released. Without a doubt, encapsulation of dorzolamide in a liposome results in a formulation capable of sustained drug delivery.

### Example 6

### Preparation of Acetazolamide and Methazolamide-Loaded Liposomes

Acetazolamide and methazolamide, like dorzolamide, are both carbonic anhydrase inhibitors used to treat open-angle glaucoma. They share a similar mechanism of action with dorzolamide, which acts to decrease aqueous humor secretion.

The thin film hydration method described above in EXAMPLE 2 was used to formulate acetazolamide- and methazolamide-loaded liposomes, termed LipoACE and LipoMET, respectively. LipoACE and LipoMET were then analyzed for drug content, mean vesicle size, and polydispersity. The results are shown in TABLE 5 below.

TABLE 5
Physical properties of LipoACE and LipoMET

| Properties | LipoACE | LipoMET |
|---|---|---|
| drug content (mg/mL) | 11.25 | 10.7 |
| mean vesicle size (nm) | 256 | 274 |
| PDI | 0.698 | 0.571 |

The results indicated that both acetazolamide and methazolamide can be incorporated into liposomes.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A stable liposomal formulation for ocular delivery, the formulation comprising a liposome including a lipid bilayer that contains a phosphatidylcholine and a polyethylene glycol-conjugated phospholipid, and a carbonic anhydrase inhibitor encapsulated in the liposome, wherein a weight ratio between the carbonic anhydrase inhibitor and the phosphatidylcholine is 1:10 to 1:100, and the liposome has a diameter of less than 2 μm and is free of cholesterol and a non-ionic detergent.

2. The stable liposomal formulation of claim 1, wherein the carbonic anhydrase inhibitor is dorzolamide, acetazolamide, brinzolamide, or methazolamide.

3. The stable liposomal formulation of claim 2, wherein the liposome has a diameter of 100 nm to 400 nm.

4. The stable liposomal formulation of claim 2, wherein the phosphatidylcholine is egg phosphatidylcholine (EggPC), palmitoyl oleoyl phosphatidylcholine (POPC), 1,2-dimyristoyl-sn-glycero-3-phosphocholine (DMPC), or 1,2-dioleoyl-sn-glycero-3-phosphocholine (DOPC) or mixtures thereof.

5. The stable liposomal formulation of claim 4, wherein the liposome has a diameter of 100 nm to 400 nm.

6. (canceled)

7. The stable liposomal formulation of claim 1, wherein the liposome has a diameter of 100 nm to 400 nm.

8. The stable liposomal formulation of claim 1, wherein the polyethylene glycol-conjugated phospholipid is 1,2-distearoyl-sn-glycero-3-phosphoethanolamine-N-[amino(polyethylene glycol)], 1,2-dioleoyl-sn-glycero-3-phosphoethanolamine-N-[methoxy(polyethylene glycol)], 1,2-dipalmitoyl-sn-glycero-3-phosphoethanolamine-N-[methoxy(polyethylene glycol)], or 1,2-dimyristoyl-sn-glycero-3-phosphoethanolamine-N-[methoxy(polyethylene glycol)] or mixtures thereof.

9. The stable liposomal formulation of claim 8, wherein the liposome has a diameter of 100 nm to 400 nm.

10. The stable liposomal formulation of claim 8, wherein each of the polyethylene glycol moieties has a molecular weight of 350 to 3000 g/mol.

11. The stable liposomal formulation of claim 10, wherein the liposome has a diameter of 100 nm to 400 nm.

12. The stable liposomal formulation of claim 8, wherein the polyethylene glycol-conjugated phospholipid is 1,2-distearoyl-sn-glycero-3-phosphoethanolamine-N-[amino(polyethylene glycol)], the polyethylene glycol has a molecular weight of 2000 g/mol, the phosphatidylcholine is POPC, the carbonic anhydrase inhibitor is dorzolamide, the weight ratio between the dorzolamide and the POPC is 1:10 to 1:30, and the liposome has a diameter of 150 nm.

13. A method for treating an ocular disorder, comprising administering to an eye of a subject in need thereof the stable liposomal formulation of claim 1.

14. The method of claim 13, wherein the ocular disorder is ocular hypertension or glaucoma.

15. The method of claim 14, wherein the stable liposomal formulation is administered via subconjunctival injection.

16-18. (canceled)

19. The method of claim 13, wherein the polyethylene glycol-conjugated phospholipid is 1,2-distearoyl-sn-glycero-3-phosphoethanolamine-N-[amino(polyethylene glycol)], the polyethylene glycol has a molecular weight of 2000 g/mol, the phosphatidylcholine is POPC, the carbonic anhydrase inhibitor is dorzolamide, the weight ratio between the dorzolamide and the POPC is 1:10 to 1:30, and the liposome has a diameter of 150 nm.

20. The method of claim 19, wherein the ocular disorder is ocular hypertension or glaucoma.
